(19) United States
(12) Patent Application Publication
(10) Pub. No.: US 2010/0030266 A1
(43) Pub. Date: Feb. 4, 2010

(54) POSTERIOR ELEMENT RIGID RETENTION SYSTEM AND METHODS OF USING SAME

(76) Inventor: Michael J. MARTIN, Federal Way, WA (US)

Correspondence Address: KENYON & KENYON LLP, 1500 K STREET N.W., SUITE 700, WASHINGTON, DC, 20005, US

(21) Appl. No.: 12/182,627

(22) Filed: Jul. 30, 2008

(51) Int. Cl. A61B 17/70

(52) U.S. Cl. 606/246; 606/278; 606/254

(57) ABSTRACT

A rigid fixation system for stabilizing the posterior element(s) of the spine is provided. The system includes a components that surround a spinous process of the affected vertebra(e). The system also includes a component that allows the assembled system to articulate to allow better positioning of the assembly and to provide maximum purchase with the bony surface of the spinous process. A method of stabilizing posterior element(s) of the spine is also provided.

### FIELD OF THE INVENTION

The present invention relates to systems and methods for rigidly fixating a posterior element(s) of a damaged vertebra.

### BACKGROUND

The spinal condition spondylolysis refers to a “crack” or defect in a portion of the vertebra called the pars interarticularis (the “pars). Anatomically, the pars is a transitional region between the anteriorly situated vertebral body and the posteriorly positioned facet joints and spinous process. Mechanically, it is an area of stress concentration between the predominantly compressive forces seen anteriorly and the tensile forces seen posteriorly. These factors in combination with the cortical type of bone present in the pars result in failure of the bone and a crack or defect that is unlikely to heal. This condition is quite common, occurring in 4-6% of the population. The disconnect between the anterior and posterior portions of the vertebra can lead to a forward migration of the vertebral body, termed spondylolytic spondylolisthesis. Clinically, spondylolytic spondylolisthesis may cause back and leg pain and may ultimately be treated by fusing the affected vertebra to the one below it. In situations where there is no spondylolisthesis, only symptomatic spondylolysis, fusion of the 2 vertebra together is considered excessive and unnecessary.

Further, there are other disadvantages to fusion as well. For example, fusion can cause the mobility of the motion segment to be transferred to other motion segments in the spine. This added stress transferred to motion segments neighboring the fused segment can cause or accelerate degeneration of those segments. Thus while fusion procedures have been used for some time, the inherent design has often caused stress concentrations and have directly and indirectly contributed to the degeneration of the joints above and below the fusion site.

There are other methods to repair the lytic defect thereby eliminating the pain generator while avoiding fusing adjacent vertebra together. Repair of the lytic defect for spondylolysis with or without spondylolisthesis can be accomplished in several ways. They all involve debridement of the defect followed by bone grafting. The loose posterior elements may then be immobilized to the vertebral body to facilitate boney union. Certain methods for securing the posterior elements to the remainder of the body include a wire or cable passed anterior to the transverse processes and inferior to the spinous process as described by Buck and a similar technique in which the wire/cable is passed around or through the heads of pedicle screws instead of around the transverse processes. (See Buck J E., “Direct repair of the defect in spondylolisthesis: Preliminary report,” Bone Joint Surg 1970, 52-13:432-7). The posterior elements may also be fixed with screws passing from distal to proximal, from the distal lamina perpendicularly through the defects, and into the posterior pedicles. None of these methods provides the ideal combination of maximal boney surfaces available for healing and rigid fixation. The cable constructs provide relatively more surface area but the construct is not rigid. The paired screws across the lytic defects provide rigid fixation but at the expense of area available for fusion/healing.

Therefore, there is a need for rigid fixation of the posterior elements to the anterior while maximizing the area available for boney healing without vertebral fusion.

### SUMMARY OF THE INVENTION

The present invention is directed to spinal fixation systems and methods of using the same whose components can be implanted on a vertebra to rigidly fixate damaged posterior element(s) of the spine. In a preferred embodiment, all components of a system are assembled on a single vertebra. An exemplary system comprises an articulation device, elongated rods and a cap piece. When locked together, certain components of the articulation device, the elongated rods, and the cap piece surround and essentially enclose a spinous process of a vertebrae as shown in FIG. 25.

During installation, the cap piece of a spinal fixation system is seated on the superior surface of the spinous process and has apertures on a bottom surface to accept the superior ends of the elongated rods as described in more detail below. The articulation device in certain embodiments comprises two arms that extend in a craniolateral direction from a base portion. In an applied position, the base portion lies inferior to the spinous process of the vertebra being treated substantially opposite the cap piece. The two free ends of the arms are connected to fastening means, such as, for example, pedicle screws that are threaded into the pedicles of the vertebra.

The articulation device further comprises articulating appendages that extend from the base portion of the articulation device. The articulating appendages articulate in the sense that they can pivot about a horizontal axis that passes through the center of the base portion. Such articulation can be accomplished through various means including via a joint that permits motion. As such, the term “pivot” as used herein with respect to the articulating appendages as well as other embodiments is used to cover all types of joints that permit motion as well as other mechanisms by which the position of a component of a system of the present invention can change from one position to another, different position. The articulating appendages are at least partially hollowed to define internal channels that are in fluid communication with superior end openings of the articulating appendages. Each channel and respective opening is configured to receive an elongated rod. In an applied position, the elongated rods extend from the articulating appendages, are positioned on the left and right side of the spinous process, and connect the articulation device to the cap piece. Depending on the height of the spinous process, the elongated rods can protrude an adjustable distance from the superior end openings of the articulating appendages to connect to the cap piece by virtue of the elongated rods' ability to pass lengthwise variable distances within the internal channels. In preferred embodiments, the top ends of the elongated rods are bulbous to allow the cap piece to tilt and maximize purchase with the superior surface of the spinous process.

The articulating appendages' ability to articulate provides the assembly with flexibility, allowing the elongated rods to be oriented at different points along the anterior-posterior axis and the inferior-superior axis to more easily capture the cap piece.

In particular, in an embodiment, a posterior element spinal fixation system comprises an articulation device comprising a connectable member having a base portion and a pair of first and second arms extending in a craniolateral direction from the base portion. The articulation device further comprises a pair of first and second articulating appendages comprising either (a) at least a top piece and a bottom piece or (b) a single one-piece. In the former embodiment, the bottom piece is connected to the base portion and the top and bottom piece are pivotably connected to each other. In the latter embodiment, the single piece is pivotably connected to the base portion. Either way, the pair of articulating appendages extend in a cephalic direction from the base portion. Each of the pair of articulating appendages comprises a shaft having an inferior end disposed against the base portion of the connectable member and a superior end opening in fluid communication with an internal channel extending at least partially through the shaft. The system further comprises a pair of first and second elongated rods each having a top end and a bottom end, each elongated rod receivable by the respective internal channel of the articulating appendage and moveable lengthwise in the internal channel. The system further comprise a cap piece having an inferior surface defining first and second openings adapted to receive the respective top ends of the pair of first and second elongated rods.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a perspective view of an embodiment of a posterior element rigid fixation system

FIG. 2 is a perspective view of the posterior element rigid fixation system of FIG. 1 in a resting position.

FIG. 3A is a perspective view of a one-piece connectable member of a posterior element rigid fixation system according to an embodiment of the present invention.

FIG. 3B is a perspective view of a multi-piece connectable member of a posterior element rigid fixation system according to an embodiment of the present invention.

FIG. 4 is a plan view of a connectable member of a posterior element rigid fixation system according to an embodiment of the present invention.

FIG. 5 is a plan view of a connectable member where the arms of the connectable member are connected to the base portion of the connectable member by a joint that permits motion according to an embodiment of the present invention.

FIG. 6 is a plan view of the connectable member of FIG. 5 where one of the arms has been angled inwards in the medial direction.

FIG. 7 is a perspective view of a two-piece connectable member of a posterior element rigid fixation system where one side of the base portion of the connectable member has a yolk that is accepted by a bore on the other side of the base portion according to an embodiment of the present invention.

FIG. 8 is a perspective view of a two-piece connectable member of a posterior element rigid fixation system where one side of the base portion has a yolk that extends through an exit orifice on the other side of the base portion according to an embodiment of the present invention.

FIG. 9 is a perspective view of a two-piece connectable member of a posterior element rigid fixation system where the bore on one side the base portion is longer than the bore illustrated in FIG. 7 according to an embodiment of the present invention.

FIG. 10 is a perspective view of a multi-piece connectable member of a posterior element rigid fixation system where each of the arms has a telescoping extension according to an embodiment of the present invention.

FIG. 11 is a perspective view of an articulation device of a posterior element rigid fixation system according to an embodiment of the present invention where the articulating appendages are two pieces and the top pieces of the articulating appendages are tilted upwards.

FIG. 12 is a perspective view of the articulation device of FIG. 11 where the articulating appendages are tilted downwards and are in a resting position such that would lie flush with a flat surface if placed thereon.

FIG. 13 is a perspective view of a partial articulation device where the bottom pieces of the articulating appendages are connected to the base portion by being integral therewith.

FIG. 14 is a perspective view of an articulation device of a posterior element rigid fixation system according to an embodiment of the present invention where the articulating appendages are each one-piece and are tilted upwards.

FIG. 15 is a is a perspective view of the articulation device of FIG. 14 where the articulating appendages are tilted downwards.

FIG. 16 is an exploded view of a posterior element rigid fixation system according to an embodiment of the present invention where the elongated rods have a top end that is bulbous.

FIG. 17 is the system of FIG. 16 wherein the components of the system are assembled into a posterior element fixation assembly.

FIG. 18 is the assembly of FIG. 17 where the cap piece is tilted in an anterior direction.

FIG. 19 is an exploded view of a posterior element rigid fixation system according to an embodiment of the present invention where the elongated rods have a top end that is bulbous and a bottom end that is bulbous.

FIG. 20 is the system of FIG. 19 wherein the components of the system are assembled into a posterior element fixation assembly.

FIG. 21 is the assembly of FIG. 20 where the cap piece is tilted in an anterior direction.

FIG. 22 is a perspective view of a cap piece according to an embodiment of the present invention.

FIG. 23 is a schematic illustration of a posterior view of a vertebra with pedicles screws inserted into the right and left pedicles.

FIG. 24 is a schematic illustration of a posterior view of the vertebra of FIG. 23 with a cap piece positioned on the superior surface of a spinous process.

FIG. 25 is a schematic illustration of a posterior view of a posterior element fixation assembly implanted on the vertebra depicted in FIG. 23.

FIG. 26 is a schematic illustration of a top view of a posterior element fixation system implanted on the vertebra depicted in FIG. 25.

FIG. 27 is a schematic illustration of a side view of a posterior element fixation system implanted on the vertebra depicted in FIG. 25.

### DETAILED DESCRIPTION

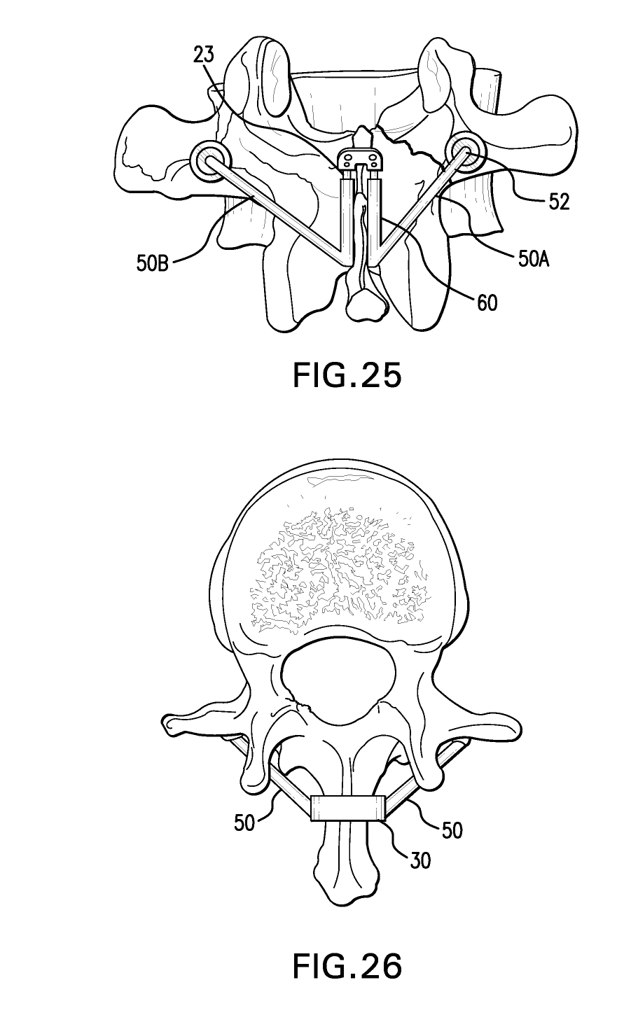

The present invention provides posterior fixation systems and methods of using the same. As used herein, the term “applied position” refer to the position and configuration of an assembly or components thereof when placed in its final, desired, fully implanted position on a vertebra(e) just prior to closing of the incision site. An exemplary illustration of a fixation system in an applied position can be seen in FIG. 25. As used herein, the term “resting position” applies to the position and configuration of an assembly, system or components thereof when such assembly, system, or components thereof are lying flat on a flat surface prior to insertion into the body. An exemplary illustration of a fixation assembly where the components are in a resting position can be seen in FIG. 2. Further, the terms “inferior,” “superior,” “anterior,” “posterior,” “upward,” “downward,” “top,” “bottom,” “horizontal,” “vertical,” “left,” “right,” “cephalic,” and “craniolateral” or vocabular modifications of such terms as well as other directional or anatomical orientation terms refer to positions and configurations of a fixation assembly in an applied position when the patient is in the anatomical position (a term well known in the art).

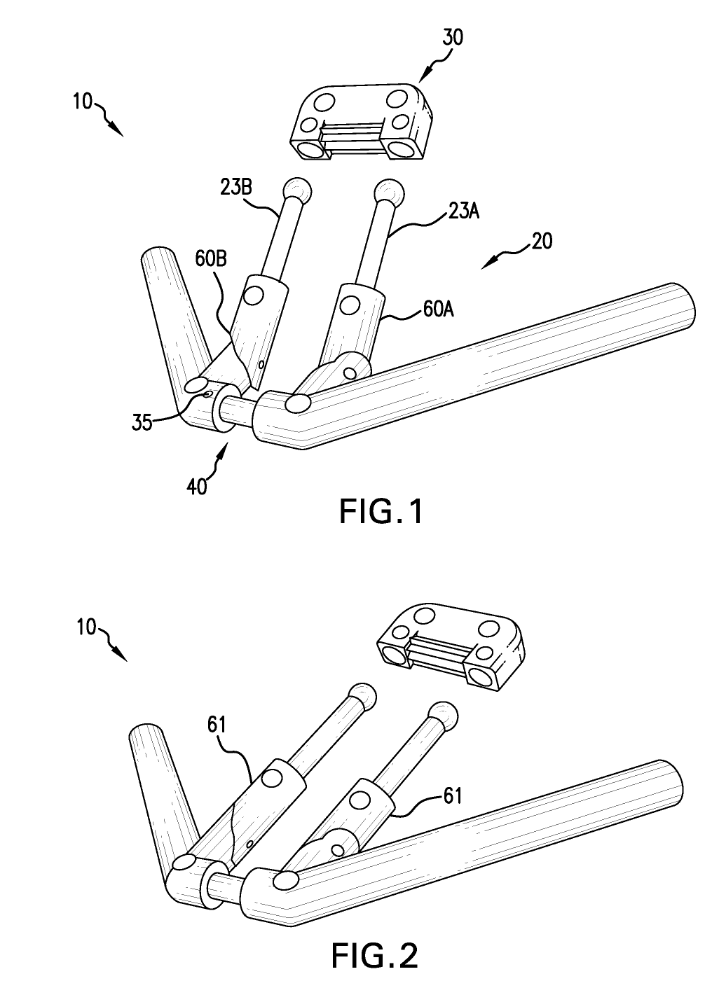

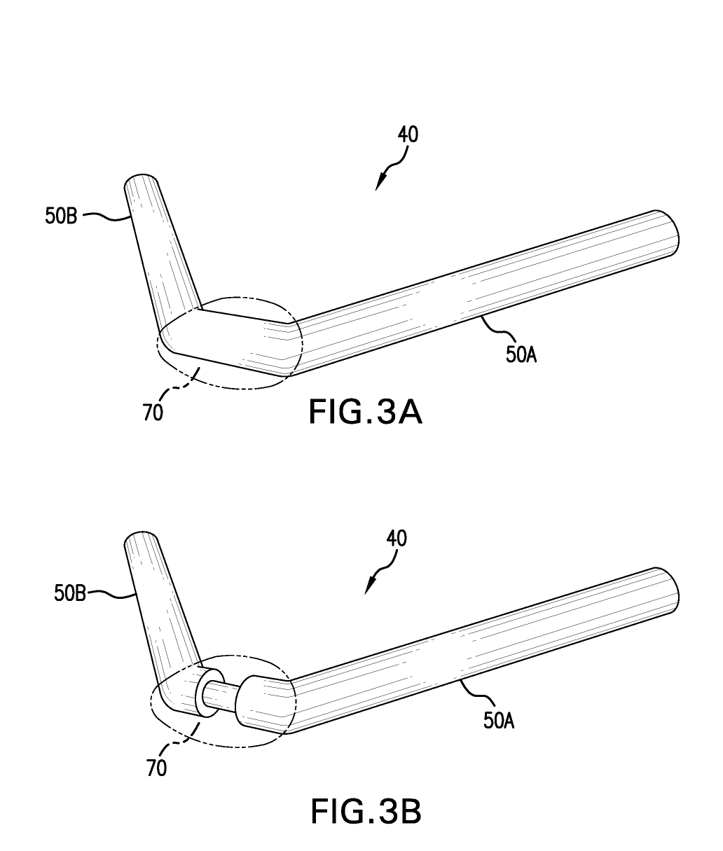

Referring to FIG. 1, an exemplary fixation system 10 comprising an articulating device 20, elongated rods 23 and a cap member 30. Articulating device 20 comprises a connectable member 40 and a pair of articulating appendages 60 extending in a cephalic direction from connectable member 40. FIGS. 3A and 3B illustrate exemplary configurations of a connectable member. As can be seen from these figures, connectable member 40 comprises a pair of arms 50A and 50B extending in a craniolateral direction from a base portion 70 of articulating device 20. For purposes of this description, arms 50A and 50B may be referred to as a first arm and a second arm, respectively, or as right and left arm, respectively.

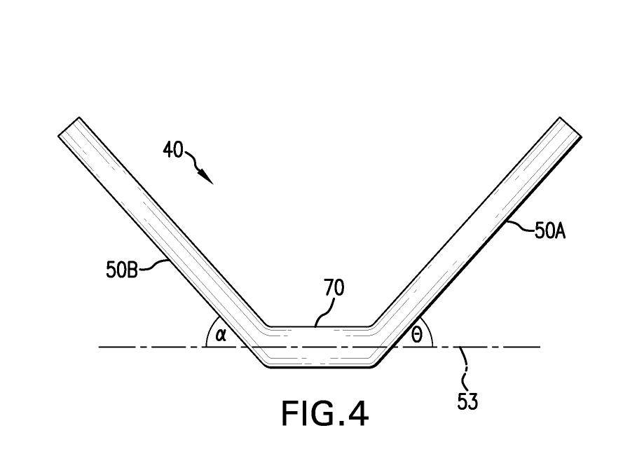

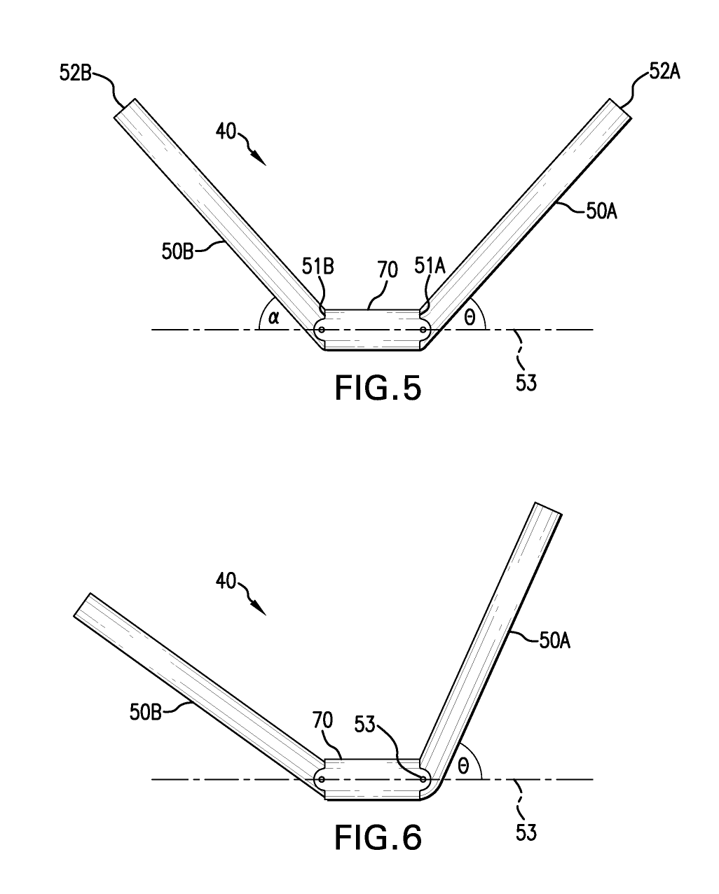

In more detail, in this exemplary configuration of connectable member 40, connectable member 40 comprises a base mount portion 70 (circled in FIGS. 3A and 3B) that transitions upward into first and second arms 50A and 50B such that the pair of arms essentially extend radially and craniolaterally from base portion 70. In a preferred embodiment, the base portion is centrally located on the connectable member. The ends of the arms that are in fluid communication with the base portion may also be referred to herein as the medial ends of the arms (referenced as ends 51A and 51B in FIG. 5) and the opposing free ends of the arms that connect to bone anchors or other fastening means in an applied position may also be referred to herein as the lateral ends of the arms (referenced as ends 52A and 52B in FIG. 5). As indicated in FIG. 4, the base portion ends and the first and second arms begin at the sections of connectable member where connectable member angles upwards. Preferably, in a resting position, these transition angles (i.e. the angles at which base portion transitions into first and second arms) (identified as angles θ and α in FIG. 4) are less than 180° but greater than 0° as measured from an imaginary horizontal line 53 passing through the center of base portion 70. In a preferred embodiment, angles θ and α are between about 45° and 70°.

In certain embodiments, the transition angle(s) θ and a between the base portion and each of the pair of arms of the connectable member can be altered during or prior to installation to accommodate different anatomical configurations of the vertebrate) being treated. For example, as shown in FIGS. 5 and 6, the medial ends 51A and 51B of connecting arms 50A and 50B can be pivotably connected to base portion 70 to allow the user to change the transition angle(s) θ and/or α. As seen by a comparison of FIGS. 5 and 6, first connecting arm 50A has been moved medially such that angle θ increases. The angled position of the arm(s) of the connectable member can be secured by placing a set screw 53 through the base portion and the arm as shown in FIG. 6 or by other fixation mechanisms. It is understood that by “pivotably connected” is meant that the arms are connected to the base portion either by a joint that permits motion or the arms are otherwise capable of being re-positioned relative to the base portion. Regarding motion joints, such joints include, for example, pivot, hinge and ball and socket joints. Regarding being otherwise capable of being re-positioned relative to the base portion, the connectable member can be made from a bendable material or a shape memory alloy or other material that allows for the transition angle θ and/or a to be altered. If made from a bendable material, the material is such that it can readily bend without breaking. Non-limiting examples of such mutable materials include titanium, titanium alloy, stainless steel and various other steel alloys.

Referring back to FIG. 3A, in certain embodiments, connectable member 40 is a “one-piece” component such that first arm 50A and second arm 50B are integral with one another at base portion 70. By “one-piece” is meant that the pair of arms are an integral, continuous, single component that are not intended to be separated from one another and cannot be detached from one another without disrupting the integrity of (i.e. breaking) the connectable member such it can no longer perform its intended function. In certain embodiments, the base portion of connectable member has an adjustable length. One way to achieve this is for connectable member to be a “multi-piece” component such that first arm 50A and second arm 50B are at least two pieces that can be connected to one another at base portion 70 as shown in FIG. 3B. By “multi-piece” is meant that the connectable member is discontinuous and has separate components that can be separated or are designed to be separated if needed without disrupting the integrity (i.e. breaking) the connectable member such that it can no longer perform its intended function. Alternatively, “multi-piece means that the connectable member is capable of being assembled from multiple parts into a single unit. For example, in certain embodiments the connectable member is “multi-piece” in that the arms are attachable to one another and can be assembled from at least two different pieces (and can, in certain embodiments, be separated or dissembled into at least two different pieces without disrupting the integrity (i.e. breaking) of the connectable member).

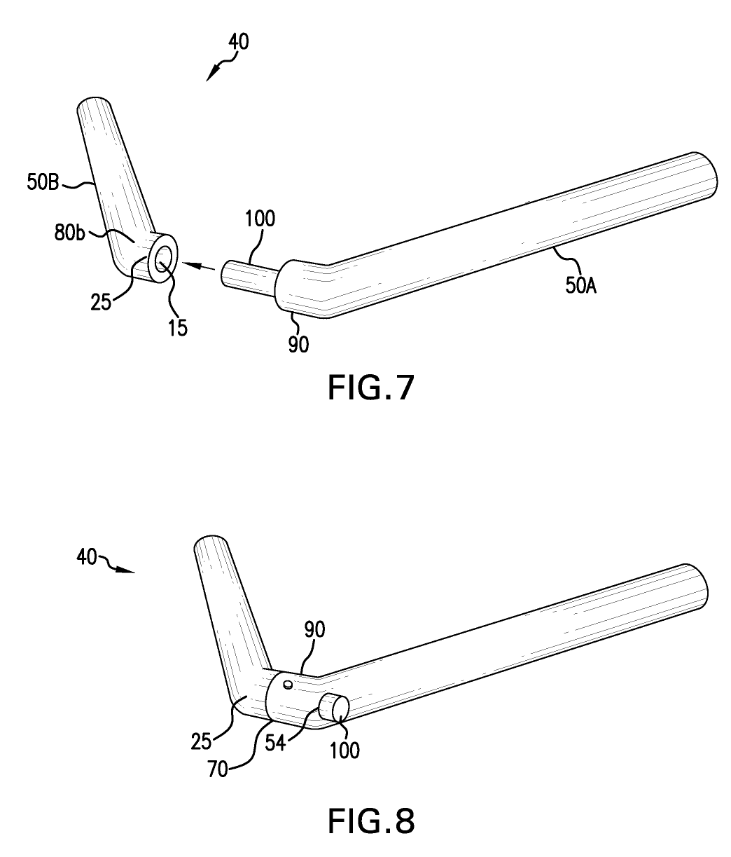

When the connectable member comprises a multiple piece component, such that the pair of connectable arms is capable of being assembled, the arms can be connected and secured together at the base portion by any fastening mechanism known in the art. For example, the arms can be coupled together by any suitable male-female fastening mechanism, an interference fit, an adhesive, threading, or a ratcheting mechanism. In one embodiment, as shown in FIG. 7, the right side 90 of base portion 70 has a yolk 100 that is acceptable by a bore 15 in the left side 25 of base portion 70. Referring back to FIG. 1, the left side of base portion 70 can define a threaded recess 35 to accept a set screw to secure yolk 100 into bore 15. Other mechanisms to secure yolk into bore 15 can also be used. Of course, it is understood that the left side of the base portion can have the yolk and the right side have the bore. In certain embodiments, base portion 70 can be adjusted to variable lengths by sliding the yolk variable distances lengthwise within the bore. In such embodiments, the length of the yolk can be such that the yolk can be inserted lengthwise variable distances within the bore. For example, as shown in FIG. 8, the right side 90 of base portion 70 can define a hollowed bore that has both an entrance orifice (not shown) and an exit orifice 54 through which a yolk 100, extending from the left side 25 of base portion 70, can pass through. Therefore, as shown in FIG. 8, when base portion 70 is at its shortest length, yolk 100 extends past or is flush with exit orifice 54. In such an embodiment, yolk 100 has a preferable length of between about 3 and 10 mm.

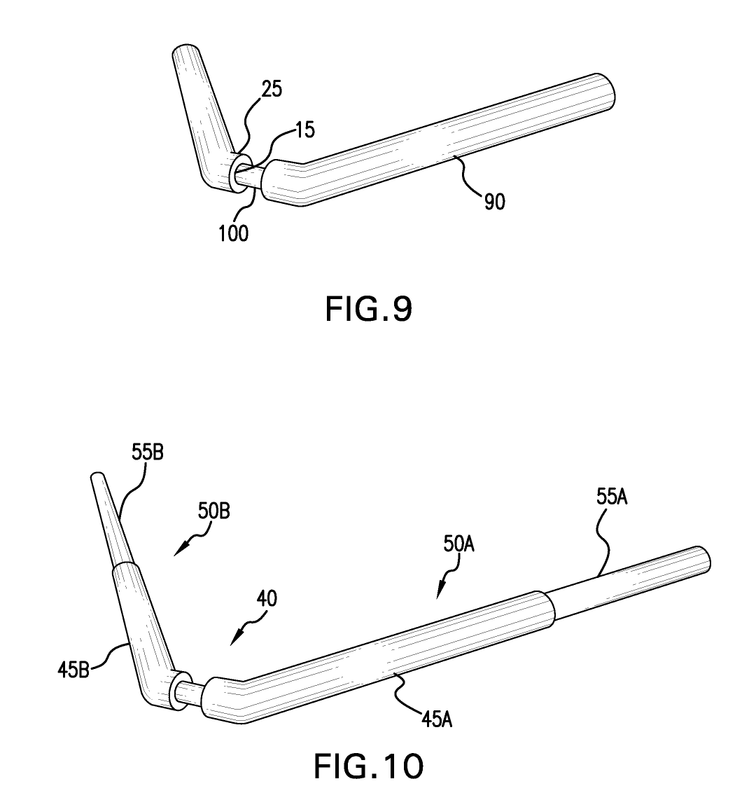

Alternatively, if it is not desired to have an exit orifice in the bore, the length of the bore can be increased such that the yolk can be inserted lengthwise variable distances within the bore without exposing the bore to an exit orifice. For example, as seen in FIG. 9, bore 15, which is defined by the left side 25 of base portion 70, can be longer than the bore as seen in FIG. 7 although the yolk 100 can be the same length. In such embodiments the length of bore 11 is preferably between about 3 and 7 mm. In either embodiment, the yolk can be locked into place via the fastening mechanisms described above or any other suitable fastening mechanism that inhibits movement of the yolk relative to the bore.

In addition or alternatively, the connectable member can be multi-piece in the sense that the base portion is capable of being assembled with the pair of arms as seen in FIGS. 5 and 6, described above, to form a connectable member.

In other embodiments, the connectable member is multi-piece in that the first and/or second arm have telescoping extensions. For example, referring to FIG. 10, in an embodiment, first arm 50A comprises a first outer sleeve 45A with an internal channel adapted to telescopically receive a first extension rod 55A and second arm 50B comprises a second outer sleeve 45B with an internal channel adapted to telescopically receive a second extension rod 55B. Therefore, if it is desired to adjust the length of one or both of the arms of a connectable member by increasing the length of one or both of the arms, the extension rod of the respective arms can be extended out a desired distance from the internal channel of the respective outer sleeve. Such an embodiment may be preferable to accommodate variable distances between the inferior portion of a spinous process and the pedicle screws that accept the lateral ends of the arms of the connectable member and that are inserted in the pedicles of a vertebra during implantation of the fixation assembly.

In other embodiments, instead of having telescoping arms, a system could include more than one connectable member with each having a different length for the arms (for example, a small, medium and large connectable member). In this way, the user could choose which connectable member to implant depending on the particular anatomy of the patient.

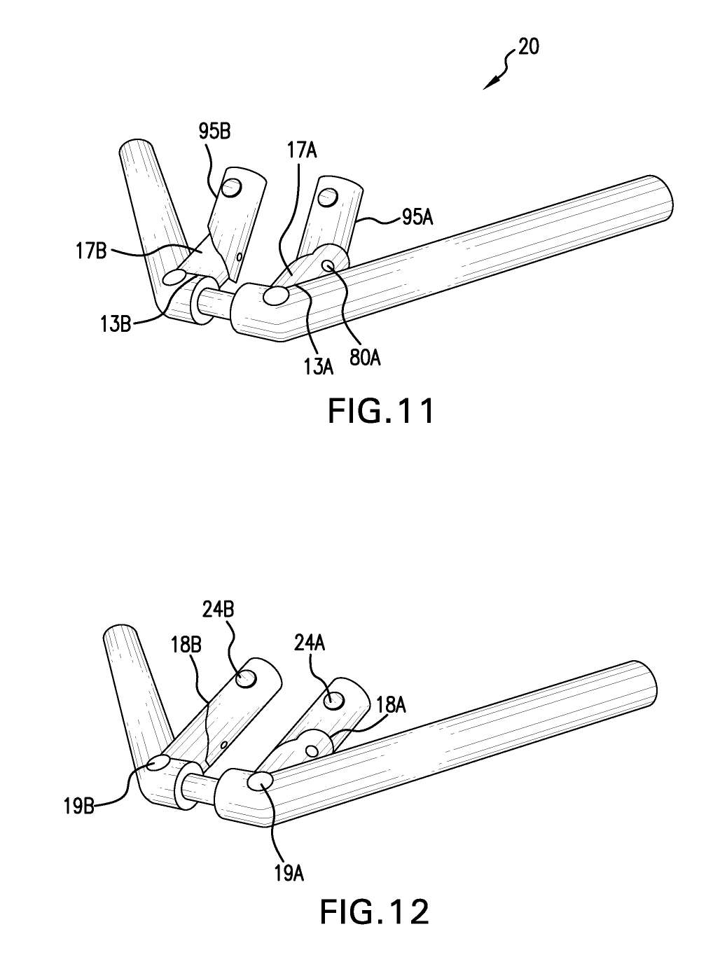

Referring to FIGS. 2 and 11, articulating device 20 further comprises a pair of articulating appendages 60A and 60B that are connected to the base portion. For purposes of this description, the pair of articulating appendages will be referred to as first articulating appendage 60A and second articulating appendage 60B or a right articulating appendage and a left articulating appendage respectively. The articulating appendages are capable of having a range of motion relative to the base portion. Specifically, the articulating appendages are capable of pivoting from a first position to a second, different position relative to the base portion.

Each articulating appendage comprises a shaft 61 having an inferior end connected to the base portion and a superior end opening in fluid communication with an internal channel extending at least partially through the shaft. The opening at the superior end is aligned with the internal channel such that the longitudinal axis of the channel passes through the superior end opening. The articulating appendages are preferably offset from one another. By preferably being offset from one another, the articulating appendages do not touch and have some distance greater than 0 separating them. This distance can be adjustable as described above if the length of the base portion of connectable member 40 is adjustable. If a fixed distance exists between the two articulating appendages, in a preferred embodiment, this distance is between about 4 and 12 mm.

As can be seen from FIG. 11, both the first and second articulating appendage extend in a cephalic, upward direction from the base portion of the connectable member. In embodiments where the base portion of the connectable member is centrally located, the pair of articulating appendages will also be centrally located on articulating device 20 by virtue of their position on the base portion of the connectable member. As shown by a comparison of FIGS. 11 and 12, the articulating ability of articulating appendages 60A and 60B allows the articulating appendages to pivot about the horizontal axis of the base portion of the connectable member (shown in FIGS. 4, 5 and 6 and referenced by character 53) to provide more flexibility in attaching cap piece 30 to articulating device 20 during implantation of the system. The articulating appendages can be pivotably connected to the base portion via a joint that permits motion. Such a joint can be any joint that permits at least lateral motion such as, for example, a pivot, a hinge, or a ball and socket joint. The articulating appendages could also be pivotably connected by being fabricated from a material that allows the articulating appendages to bend (as described above with respect to the arms of the connectable member) in the anterior to posterior direction as well as the inferior to superior direction.

An articulating appendage can be a single integral piece or a multi-piece unit comprising at least two pieces that communicate with each other. For example, in certain embodiments, as shown in FIGS. 11 and 12, each articulating appendage comprises a two-piece unit comprising a top piece 95 and a bottom piece 17. In these embodiments, each top piece is pivotably connected to the respective bottom piece with respect to the base portion to allow each articulating appendage to pivot about the horizontal axis of the base portion. In particular, in the embodiments illustrated in FIGS. 11 and 12, bottom pieces 17A and 17B have inferior ends 13A and 13B that are connected to base portion 60 of connectable member 40, and superior ends 18A and 18B that are pivotably connected to the respective inferior ends of top pieces 95A and 95B, the latter connection forming the joints of the articulating appendages. The top and bottom pieces can be pivotably connected in various different ways so long as the final relative position of each top and bottom pieces can be locked in place. In particular, the bottom pieces of the articulating appendages can define set screw holes 80A and 80B (not shown) that can be threaded recesses for set screws to lock the joints once the device is in a desired application position. Of course other locking mechanism could also be used.

Regarding the top and bottom pieces being pivotably connected in various ways, the top and bottom pieces can form a ball and socket joint with either the top piece having a bulbous inferior end and the respective bottom piece having a superior end shaped like a socket or the bottom piece having a bulbous superior end and the respective top piece having an inferior end shaped like a socket. Such a ball and socket joint allows rotational movement of the top piece of the articulating appendage relative to the bottom piece as well as lateral movement. Such a joint could also be locked in place with a set screw or an adhesive or other locking or securement mechanism. Other joints that permit motion include hinge joints. The articulating appendages also can be pivotably connected to the base portion by being fabricated from a bendable material as described above with respect to the arms of the connectable member.

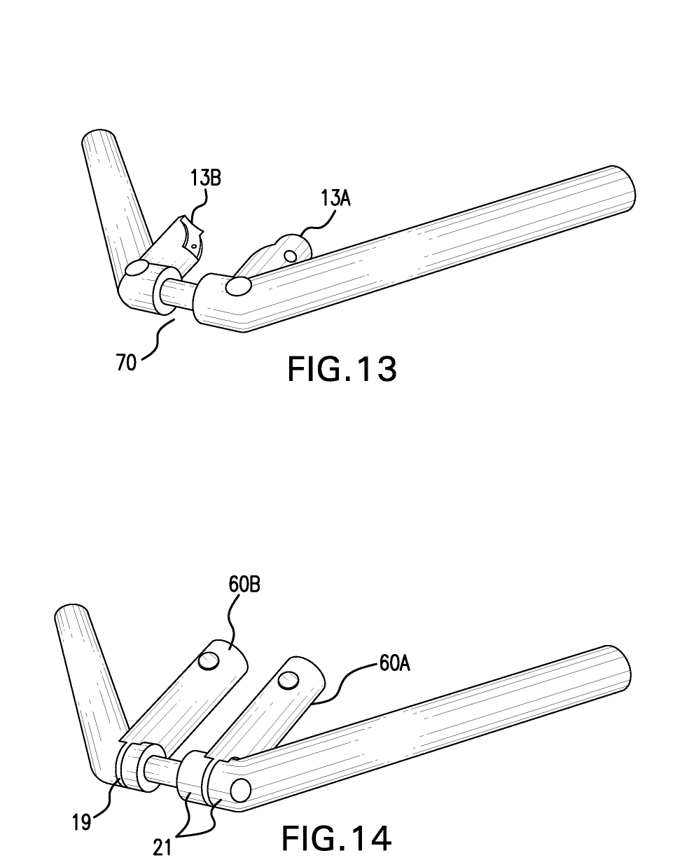

The bottom pieces 17 of articulating appendages 60 can be an integral, continuous section of the connectable member or can be a non-integral discontinuous attachment to the connectable member. Regarding the former, as shown in FIG. 13, the inferior ends 13A and 13B of the bottom pieces are connected to the base portion such that the inferior ends are integral with the base portion 70 of connectable member 40. In other words, there is no discontinuity between base portion 70 and the inferior ends of bottom pieces 13A and 13B and the bottom pieces are in fluid communication with the base portion. In such embodiments, the base portion is likely manufactured together with the bottom pieces by injection molding or other manufacturing methods that allow the base portion to transition into the bottom pieces of the articulating appendages. Alternatively, as shown in FIG. 11, the inferior ends of bottom pieces 17A and 17B can be a connected to the base portion such that the bottom pieces are distinct separate structures from the bottom portion and there is discontinuity between the base portion and the inferior ends of the bottom pieces. In such embodiments, the bottom pieces are likely manufactured separately from the connectable member and attached to base portion of connectable member during a later stage of manufacturing. For example, as illustrated in FIG. 12, bottom piece 17A is connected to base portion 70 by a screw 19A and bottom piece 17B is connected to base portion 70 by a screw 19B. In any event, articulating appendages can be connected to the base portion of the connectable member by any suitable mechanism including, for example, threadable attachment or other ways of fixed connection.

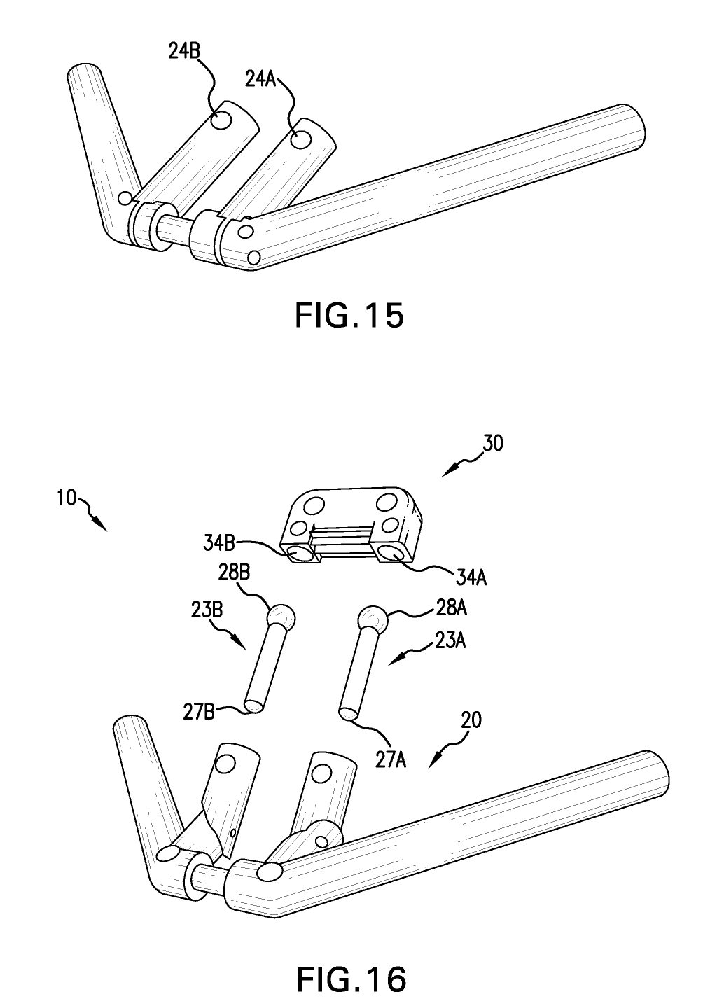

As mentioned above, an articulating appendage can be a single integral piece. For example, referring to FIG. 14, articulating appendages 60 are both one-piece structures that are pivotably connected to base portion 70 of connectable member 40. As with the two-piece embodiment each articulating appendage can pivot about the horizontal axis of base portion 70 as shown by a comparison of FIGS. 14 and 15. In particular, in this embodiment, articulating appendage 60 and the base portion are connected via a hinge that is formed by a pin-like component 19 of articulating appendage 60 that is sandwiched between a collar 21 in base portion 70. The pin and collar allow for lateral movement of the articulating appendages.

Therefore, the articulating appendages provide the fixation system with flexibility such that the user can pivot and orient the articulating appendages at various angles to reposition the articulating appendages such that they mate with the cap piece at a proper orientation. Once the proper orientation is achieved, the articulating appendages can be rigidly fixed in place by any suitable fixation means (including, as described above, an adhesive or a set screw) such that the joint does not move.

Referring back to FIG. 1, fixation system 10 further includes a pair of elongated rods 23A and 23B that are received at one end by the superior end openings and internal channels of the articulating appendages and received at another end by cap piece 30. As such, the elongated rods allow connection between articulation device 20 and cap piece 30. The internal channels of the articulating appendages allow the elongated rods to move lengthwise within the articulating appendages which, in turn, allow the elongated rods to protrude a desired distance from the superior end openings of the articulating appendages. Of course, such distance can be adjustable by adjusting the depth in which the elongated rods are slid, threaded or otherwise moved lengthwise into the internal channels of the articulating appendages. The superior portions of articulating appendages 60 can define threaded recesses 24A and 24B as shown in FIG. 15 for set screws to lock the elongated rods in place a certain desired, fixed distance between the articulation device and the cap piece.

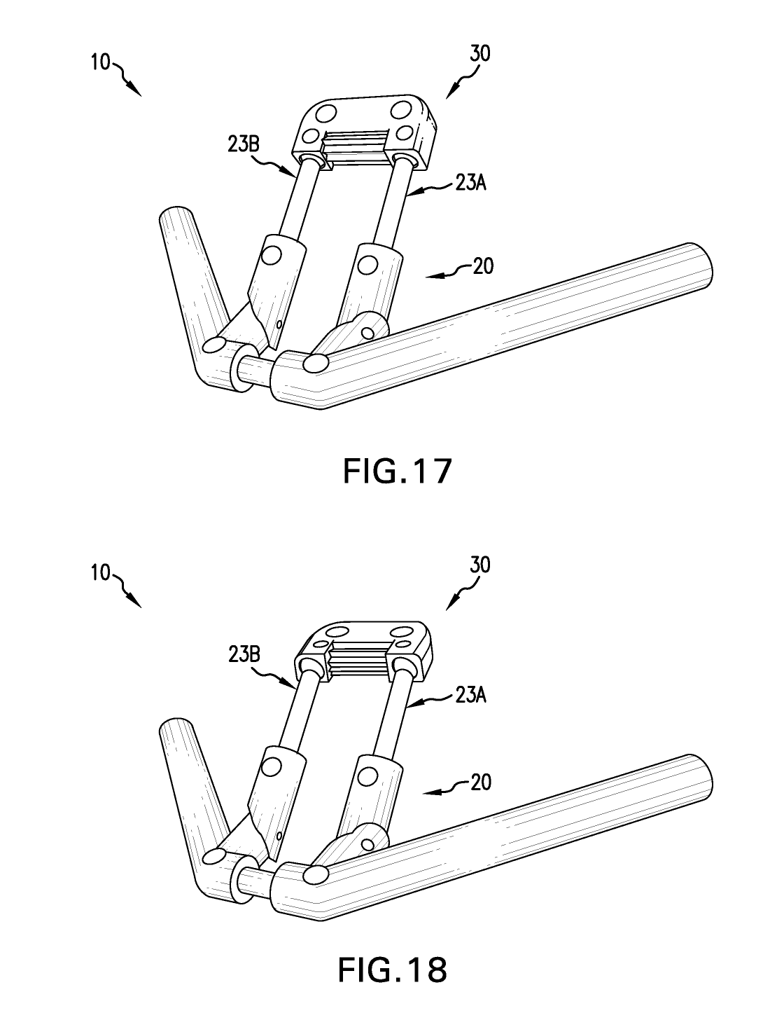

As shown in FIG. 16, in certain embodiments, each elongated rod 23 comprises a bottom end 27 and a top, preferably bulbous end 28. The bulbous ends allow for rotational as well as linear motion of the elongated rods relative to the cap piece to better adjust to the position of cap piece 30 when the fixation system is being applied to a vertebra. The bulbous ends also allow the cap piece, one attached to the elongated rods, to tilt to better purchase the bone of the spinous process. Specifically, referring to FIGS. 17 and 18, the bulbous ends 28 are captured by openings 34 defined by the inferior surface of the cap piece (described in more detail below). As shown by a comparison of FIGS. 17 and 18, the bulbous ends allow cap piece 30 to tilt in an anterior to posterior direction as well as an inferior to superior direction to allow better positioning of cap piece 30 to maximize purchase with the superior surface of the spinous process during installation. It should be noted that the cap piece shown in FIG. 18 may be an exaggerated image to show the cap piece in a different position than in FIG. 17. During installation, the cap piece may not need to tilt back as anteriorly as shown in FIG. 18.

As mentioned above, the elongated rods are slidable or otherwise moveable lengthwise in a channel extending at least through the superior section of the top portion or top piece of the articulating appendages and such movement allows for adjustment of the distance the elongated rods protrude from the top portion or top piece of the articulating appendage. Such adjustability, in turn allows for adjusting how high the cap piece is placed during installation. The elongated rods can be moveable in a lengthwise direction by being in slidable or threadable communication with the articulating appendages or by other means which allows the distance the elongated rods protrude to be adjusted and locked in position once the distance is desired to be fixed.

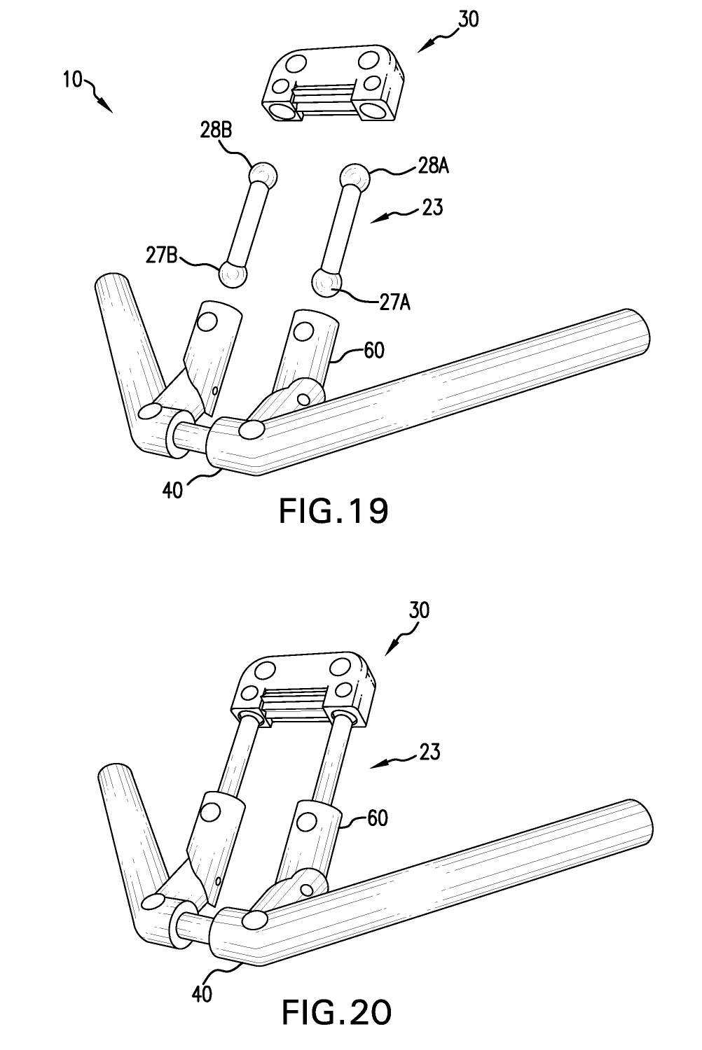

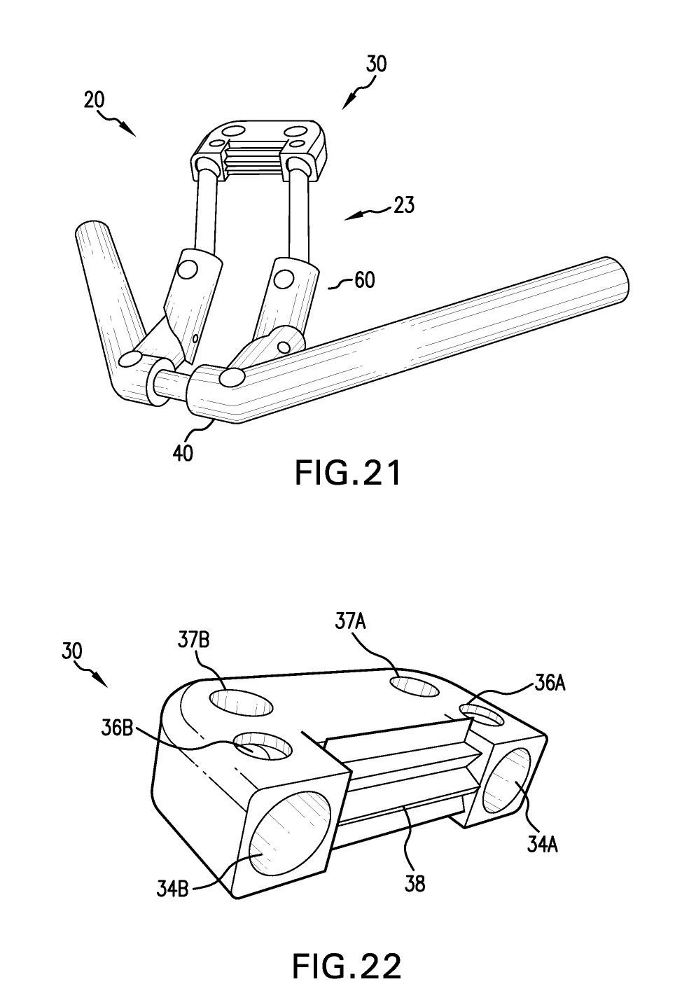

Referring to FIG. 19, in certain embodiments, bottom ends 27 and top ends 28 of elongated rods 23 are both bulbous. Such a configuration of elongated rods 23 confers lateral motion both at the top and bottom ends of rods 23 as shown by a comparison of FIGS. 20 and 21. It is also within the present invention for only top or bottom ends to be bulbous.

The cap piece will now be described in more detail. As described above, the cap piece is connectable to the elongated rods 23A and 23B, which are, in turn, received by the pair of first and second articulating appendages 60A and 60B, respectively. The cap piece is configured to sit on and capture a superior surface of the spinous process of the vertebrae being treated. The cap piece has a length such that it extends past the lateral sides of the spinous process to expose two openings on a bottom surface of the cap piece that are configured to receive the top ends of the elongated rods. The adjustability of the articulating appendages 60A and 60B and elongated rods 23A and 23B maximizes the “bite” and bony interface for the cap piece with the superior aspect of the spinous process.

The details of an exemplary cap piece are illustrated in FIG. 22. Openings 34A and 34B can be invaginated (drilled out) receptacles to accept the top ends of elongated rods 23A and 23B, respectively. Set screw holes 36A and 36B can be threaded recesses for set screws to lock elongated rods 23A and 23B in position. Of course, other fixation mechanisms to lock elongated rods in place relative to cap piece 30 can also be employed. Apertures 37A and 37B can act as openings to accept an insertion device for installing the device. Although apertures 37 are shown located on the superior surface of the cap piece, the apertures could be located on other surface as well. Cap piece 30 can have contact portion 38 on the inferior surface thereof that comprises a horizontally sharp-ridged, serrated surface for interfacing with the bone of the spinous process. Of course, contact portion 38 can have any suitable surface, including bumpy, for example, that frictionally prevent cap piece 30 from sliding off of the spinous process when in an applied position.

The various components of a fixation system according to the present invention can be fabricated from the same or different materials. The materials could be any metal appropriate for surgical implantation such as stainless steel, titanium, titanium alloy, chrome alloys, such as nickel titanium. In addition, the fixation system may be formed from non-metallic materials including but not limited to, carbon fiber, resin materials, plastics and ceramics. In certain embodiments, the fixation system is fabricated from a bioresorbable material including, but not limited to, silicone, polyurethane, polyester, polyether, polyalkene, polyamide, poly(vinyl)fluoride, polytetrafluoroethylene (PTFE), glass, carbon fibers, and suitable mixtures thereof. In addition, composite materials, such as a matrix of fibers, may be used to form at least a portion of the fixation system. In all embodiments, the components of the fixation system are sterile and biocompatible.

The bone fasteners that are inserted into the vertebra(e) as described above and that accept the lateral ends of the arms of a connectable member can be any type of fastener that may be attached to the arms while remaining securely fastened to the intended bone. Thus the bone fasteners may include polyaxial screws, helical blades, expandable screws, such as Mollic bolt type fasteners which are inserted or screwed into the bone and expand by way of an expansion mechanism, conventional screws, staples, sublaminar hooks and the like.

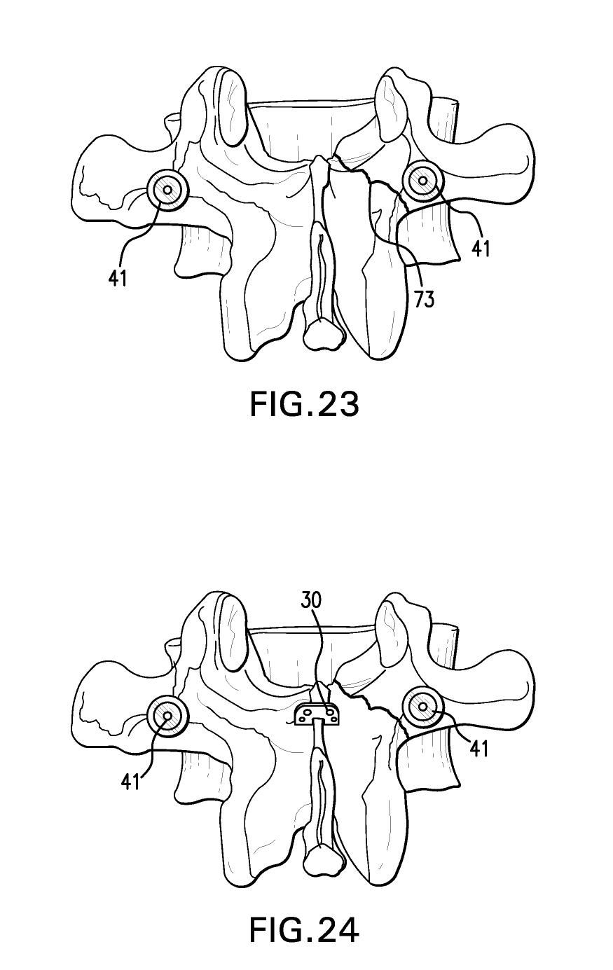

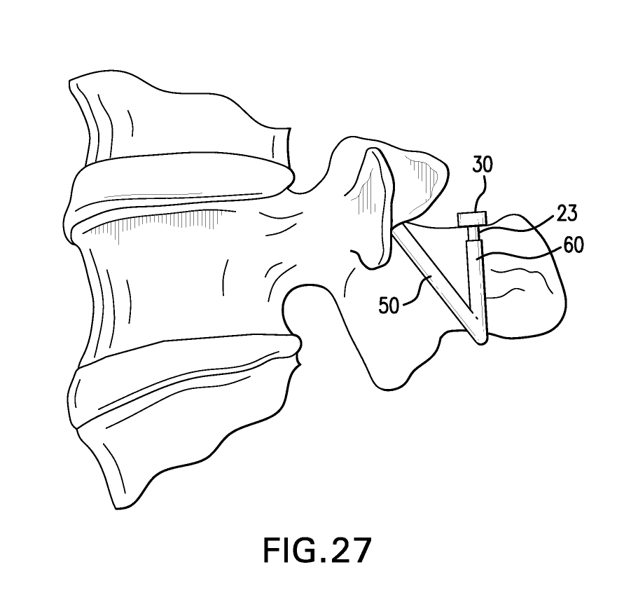

The present invention also provides a method for rigidly stabilizing the posterior elements of a damaged vertebra. An exemplary method will be described with reference to FIGS. 23-25. Referring to FIG. 23, pedicle screws 41 are inserted into the vertebra desired to be treated. Reference character 73 indicates a crack in the pars of the vertebra. Of course other pathological conditions could also be treated by methods of the present invention. Referring to FIG. 24, cap piece 30 is placed on the superior surface of the spinous process such that the cap piece crosses over the spinous process. Referring to FIG. 25, the base portion 70 of connectable member 40 is positioned inferior to the spinous process opposite cap piece 30. Lateral ends 52 of arms 50 are secured to pedicle screws 41. Articulating appendages 60 are adjusted to the correct angle to allow elongated rods 23 to mate with cap piece 30. Cap piece 30 is adjusted in the anterior-inferior to posterior-superior direction to better capture and purchase the superior surface of the spinous process. A top view of the fixation assembly is provided in FIG. 26 to show the placement of the cap piece 30 on the superior surface of the spinous process relative to the arms 50 of the connectable member and the posterior elements of the vertebra being treated. FIG. 27 is provided to show the placement of the elongated rods and articulating appendages relative to the cap piece and the posterior elements of the vertebra being treated.

A bone graft can then be placed on the crack 73 in the pars to allow the pars to heal. The bone material used can be a bone graft material or a BMP. Bone graft materials are well known in the art and include both natural and synthetic materials. For example, the bone graft material can be an autologous or autograft, allograft, xenograft, or synthetic bone graft. BMPs are also well known in the art and include BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, BMP-15. Preferred BMPs are any of BMP-2, BMP-3, BMP-4, BMP-5, BMP-6, and BMP-7. The bone material can also include other therapeutic agents such as anti-microbial agents or antibiotics.

Posterior element retention assemblies of the present invention can be used to treat or improve spinal conditions that affect the posterior element(s) of the spine. In particular, assemblies of the present invention can be used to treat damaged vertebra(e). The vertebra(e) can be damaged, for example, as a result of disease, trauma or age-related deterioration. Non-limiting examples of diseases include spondylolysis and spondylolisthesis, posterior element fractures, joint resurfacing and adjacent segment shielding. The systems can be used on various sections of the spine including the lumbar, cervical, thoracic and/or sacral regions. In preferred embodiments, the regions are the lumbar regions. If the condition being treated is spondylolysis, the preferred region is the vertebra at level L5.

In a preferred embodiment, all the components of the fixation assembly are positioned and fixed on one vertebral segment (i.e. one vertebra) and do not span over more than one vertebral segment (i.e. more than one vertebra). Even so, the assembly can span more than one vertebrae. Alternatively, two or more fixation systems can be stacked.

An exemplary method of surgically installing the device will now be described. The posterior elements of the affected vertebra are exposed subperiosteally in the standard fashion. Dissection is far enough laterally to allow insertion of pedicle screws into the vertebra with the lytic defect in the posterior elements. Dissection continues proximally past the inferior margin of the next superior spinous process and distally past the inferior border of the affected spinous process. The lytic defect is debrided and decorticated in preparation for bone grafting and/or placement of biologics to enhance fusion of the lytic defect.

Using standard technique, pedicle screws are placed in the affected vertebra. The cap piece is positioned over the dorsal surface of the spinous process of the affected vertebra. The arms of the articulation device are assembled such that base portion will be flush beneath the inferior surface of the affected spinous process. The articulating appendages of the articulation device will be on either side of the affected spinous process. The lateral ends of the arms are attached to the previously placed pedicle screws. The elongated rods attach to the cap via their bulbous ends while the opposite ends slide into the hollowed articulating appendages. The various set screws are tightened, thereby firmly capturing the spinous process and posterior elements of the affected vertebra.

Autologous iliac crest bone graft, allograft and/or biologic fusion enhancers are placed in the previously prepared defects. The construct can then be compressed if desired. Final tightening of the assembled construct to the pedicle screws firmly fixes the posterior elements to the anterior elements of the spine.

I claim:

1. A posterior element spinal fixation system comprising:
an articulation device comprising:
a connectable member having a base portion and a pair of first and second arms extending in a craniolateral direction from the base portion;
a pair of first and second articulating appendages comprising a top piece and a bottom piece, the bottom piece connected to the base portion, the top and bottom piece pivotably connected to each other, the pair of articulating appendages extending in a cephalic direction from the base portion, each of the pair of articulating appendages comprising a shaft having an inferior end disposed against the base portion of the connectable member and a superior end in fluid communication with an internal channel extending at least partially through the shaft;
a pair of first and second elongated rods each having a top end and a bottom end, the bottom end receivable by the respective internal channel of the articulating appendage and moveable lengthwise in the internal channel; and
a cap piece having an inferior surface defining first and second openings adapted to receive the respective superior end of the pair of first and second elongated rods.

2. The fixation system of claim 1, wherein the top and bottom piece are pivotably connected to each other by a joint that permits motion.

3. The fixation system of claim 1, wherein the base portion is assembled from a separate first and second arm.

4. The fixation system of claim 1, wherein the first and second arms are at least two separate pieces detachable from one another at the base portion of the connectable member.

5. The fixation system of claim 4, wherein at the base portion either of the first or second arms comprises a male fastener and the other of the first or second arms comprises a female fastener so that the pair of arms are connectable to one another at the base portion.

6. The fixation system of claim 5, wherein either of the first or second arms comprises a yolk and the other of the first and second arms defines an internal bore configured to accept the yolk.

7. The fixation system of claim 1, wherein the base portion has an adjustable length.

8. The fixation system of claim 1, wherein the first and second arms are non-detachably connected to each other such that they form an integral one-piece member.

9. The fixation system of claim 1, wherein the transition angle is capable of being altered during use.

10. The fixation system of claim 9, wherein the first and second arms are pivotably connected to the base portion of the connectable member.

11. The fixation system of claim 10, wherein the first and second arms are fabricated from a bendable material.

12. The fixation system of claim 1, wherein the first and second arms each comprise telescoping extensions to adjust the length of the first and second arms.

13. The fixation system of claim 1, wherein the articulating appendages are centrally disposed against the connectable member of the articulating device.

14. The fixation system of claim 1, wherein the pair of articulating appendages are offset from one another.

15. The fixation system of claim 1, wherein the top ends of the elongated rods have a bulbous shape.

16. The fixation system of claim 15, wherein the bottom ends of the elongated rods have a bulbous end.

17. The fixation system of claim 1, wherein the inferior surface of the cap portion is serrated.

18. The fixation system of claim 1, wherein a surface of the cap portion has apertures to receive ends of an insertion instrument.

19. The fixation system of claim 18, wherein the surface of the cap portion that has apertures is the superior surface.

20. A posterior element spinal fixation system comprising:
a connectable member having a base portion and a pair of first and second arms extending in a craniolateral direction from the base portion;
a pair of first and second articulating appendages pivotably connected to the base portion, the pair of articulating appendages extending in a cephalic direction from the base portion, each of the pair of articulating appendages comprising a shaft having an inferior end disposed against the base portion of the connectable member and a superior end in fluid communication with an internal channel extending at least partially through the shaft;
a pair of first and second elongated rods each having a top end and a bottom end, the bottom end receivable by the respective internal channel of the articulating appendage and moveable lengthwise in the internal channel; and
a cap piece having an inferior surface defining first and second openings adapted to receive the respective superior end of the pair of first and second elongated rods, the cap piece configured to cross over a spinous process in an applied position.

21. A method of implanting a posterior element fixation assembly comprising:
inserting a left fixation means in a left pedicle of a vertebra and a right fixation means in a right pedicle of the vertebra;
placing a cap piece on the superior surface of the spinous process of the vertebra, the cap piece having a pair of opening on an inferior surface thereof;
providing an articulation device comprising:
a connectable member comprising a pair of left and right arms extending in a craniolateral direction from the base portion, the pair of left and right arms having a left and right free end respectively; and
a pair of left and right articulating appendages connected to the base portion and capable of pivoting about a horizontal axis passing through the center of the base portion, each of the articulating appendages having a shaft defining an internal channel extending at least partially through the shaft;
providing a pair of left and right elongated rods each having an inferior end and a superior end received by a respective internal channel of the pair of articulating appendages, the elongated rods capable of moving lengthwise through the channels to extend an adjustable distance from the articulating appendages;
attaching the left free end of the pair of arms to the left fixation means and attaching the right free end to the right fixation means; and
adjusting the distance and orientation the elongated rods protrude from the articulating appendages so that the superior ends of the elongated rods are captured by the openings of the cap piece.
